(19) United States
(12) Patent Application Publication
(10) Pub. No.: US 2025/0215021 A1
(43) Pub. Date: Jul. 3, 2025

(54) SYSTEMS AND METHODS UTILIZING THERANOSTIC AGENTS FOR BPCT AND BNCT FOR TARGETED CANCER TREATMENT

(71) Applicant: University of Virginia Patent Foundation, Charlottesville, VA (US)

(72) Inventors: Dongfeng Pan, Charlottesville, VA (US); Tyvin A. Rich, Charlottesville, VA (US); Mahendra D. Chordia, Charlottesville, VA (US)

(21) Appl. No.: 18/852,262

(22) PCT Filed: Apr. 5, 2023
(86) PCT No.: PCT/US2023/017586

§ 371 Date: Sep. 27, 2024

Related U.S. Application Data
(60) Provisional application No. 63/327,554, filed on Apr. 5, 2022.

(51) Int. Cl. C07F 5/02; A61K 41/00; A61K 47/54; A61K 51/04; A61P 35/00; C07B 59/00

(52) U.S. Cl. CPC C07F 5/027; A61K 41/0038; A61K 41/0095; A61K 47/545; A61K 51/0446; A61K 51/0497; A61P 35/00; C07B 59/002; C07B 59/004

(57) ABSTRACT

Boronated prodrugs have been developed that are particularly advantageous for use in boron proton capture therapy (BPCT) and boron neutron capture therapy (BNCT). Cancer-targeting moieties, such as heptamethine cyanine dyes (HMCDs), are linked to compounds including a plurality of boron isotopes, e.g., carboranes such as 1-amino-1-carbadodecaborate. Compounds of the present disclosure were demonstrated to deliver eleven boron-11 atoms per dye molecule selectively to breast cancer cells. Upon irradiation with proton or neutron beams and subsequent nuclear fusion reaction by proton capture, unstable ¹²C is generated in place of ¹¹B, which is short lived and releases high energy alpha particles. There particles travel only a short distance within cell and/or a very close tumor microenvironment, damaging cellular DNA ultimately resulting in killing cancer cells, and are particularly useful with young patients and with deep tissue tumors located in critical organs which are difficult to remove by surgery.

### CROSS REFERENCE TO RELATED APPLICATION(S)

This application claims the benefit of U.S. Provisional Application No. 63/327,554, filed Apr. 5, 2022, which is incorporated by reference as if disclosed herein in its entirety.

### BACKGROUND

Initial reports demonstrating in vitro and in vivo evidence of cancer cell selective uptake of certain heptamethine cyanine dyes (HMCDs) for imaging has spurred immense interest for targeted dispensing of anti-cancer payloads. A variety of HMCDs are known to target cancer cells selectively and many HMCD-drug conjugates and radiological probes are being explored for cancer therapeutics and diagnostics. Without wishing to be bound by theory, the selective uptake of HMCDs in cancer cells is powered by over expression of organic anion transporter proteins (OATPs). This cancer selective uptake can be augmented by micropinocytosis and hypoxic conditions of cancer cells in the tumor microenvironment.

Accordingly, in the last several years conjugates of HMCDs with various anti-cancer drug cargos, e.g., cis-platin, gemcitabine, taxol, dasatinib, methotrexate etc., and other drugs in development, e.g. monoamine-oxidase inhibitor NMI, farnesyl-thiosalicylic acid, genistein etc., have been reported. Near-infrared fluorescence dye (NIRF)-(DZ-002 statin) conjugate based therapy has been submitted for FDA IND approval for clinical studies in humans reflecting its potential to be a therapeutic agent. Indocyanine green, another NIRF belonging to the same class of HMCDs, is currently in clinical use for determining cardiac output, hepatic function, liver and gastric blood flow, and for ophthalmic angiography, indicating the safety profile of this class of molecules in humans.

Proton therapy is now a mainstay strategy in cancer treatment due to its precise delivery of energy to cancerous tumor cells in an inverted depth-dose form, thus causing low irradiation of normal tissue and fewer side effects. Also called proton beam therapy or proton capture therapy, proton therapies treat cancer utilizing high energy protons (positively charged particles) generated in a synchrotron or cyclotron. The protons travel through tissue upon irradiation and precisely deliver the energy at depths not easy to treat. Therapeutic proton beams are not as efficient as photons or electrons utilized in radiobiology. However, a focused compact path of high energy protons accurately deposits energy for fusion (atom transmutation/capture) at a distance defined by Bragg peak, thereby sparing a majority of the surrounding tissue. The proton beams can generate short-lived activated nuclei of carbon, oxygen, and nitrogen atoms in a dose and depth dependent manner. This transmutation of atoms and subsequent radiation events results in the release of highly damaging energy at high concentration in a very localized manner, causing irreversible damage to cellular organelles and compartments and cellular mortality. Another important facet of proton therapy is that physical proton capture with other nuclei can overcome cancer radioresistance and make cells sensitized towards irradiation. It has been demonstrated that cancer cell uptake of 18-labelled thymidine and its insertion in DNA causes complex DNA lesions, resulting in efficient cell killings of squamous cancer SQ20B cells. That demonstration described transmutation of ¹⁸O+p→¹⁸F and disruption of DNA structure.

Unlike gamma radiation therapy that causes huge radiation related side effects to the normal tissue through which it travels, the proton beam comes to a halt once it reaches the tumor, where it conforms to the shape and depth of the tumor and only then releases its full energy. Therefore, this type of advanced radiotherapy spares the surrounding normal tissue resulting in less side effects. Proton therapy has shown tremendous potential in treating several kinds of cancer and it is considered as the most safe and best therapy by radiation oncologists to strike tumors precisely by manipulating the proton beams.

Proton therapy is more efficacious than other radiation therapies, but its availability and utility is still in infancy compared with X-ray radiation therapy due to the high cost of development of infrastructure and required skills for this advance technology. Hence, the true potential of proton therapy on prolonging life in solid tumor patients is not yet fully realized. In the last decade there has been explosive growth in utilization of proton therapy in clinics by radio-oncologists, and over 280,000 patients (over 200 clinical trials) had been treated by the end of 2021 as reported by Particle Therapy Co-operative Group (PTCOG). See Particle Therapy Co-Operative Group. Particle Therapy Centers. Available at: www.ptcog.ch. Considering its promise and safety potential, many brand-new proton-therapy centers are being built in the United States and in countries across the world. There are over 100 proton and neutron therapy facilities in the world now treating patients with cancer. This number will continue to increase to meet the needs of the increasing burden of cancer worldwide.

In recent years, proton capture specifically by boron-11 (also referred to herein as ¹¹B) has attracted attention for anti-cancer clinical applications due to release of short-range high-linear energy transfer (H-LET) alpha particles. Compared to proton capture by boron-11, neutron capture therapy (BNCT) by boron-10 (also referred to herein as ¹⁰B) is clinically now established. One of the limitations of effective proton-boron capture therapy (BPCT) is the relative low natural abundance of boron-11 (80.2%) in cancer cells and/or its accumulation from external sources.

Even though proton capture cross section for ¹¹B is highly desirable (in 0.1-10 MeV range), its efficiency in BPCT depends on localization and concentration of boron-11 atoms in cancer cells. Moreover, the effectiveness of BPCT can depend on the incident energy of proton beam, source size, cellular array size, thickness of medium layer through which beam is travelling, etc. Boron-11 has high natural abundance (about 80.2%) and boron compounds are known to play some functional role in plant cells, but its role in mammalian cells is not well studied. In addition, pharmacodynamic and radiobiological efficiency in terms of selective delivery and accumulation of ¹¹B isotope in cancer cells and tumor ultimately poses major hurdle in achieving its clinical potential.

There is the need to improve proton and neutron treatment and decrease toxicity of treatment. A method to enhance the effectiveness of proton and neutron beam therapy to increase the chance of eradication of the cancer while sparing the effects on normal tissue is highly desired.

### SUMMARY

Aspects of the present disclosure are directed to a compound according to Formula III:

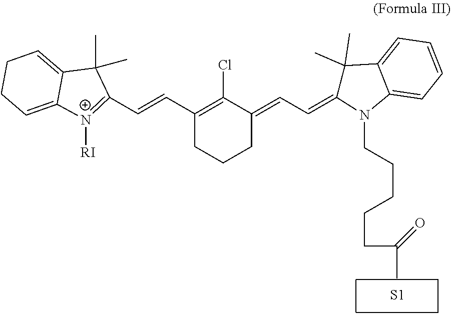

In some embodiments, S1 includes a plurality of ¹⁰B isotopes, ¹¹B isotopes, or combinations thereof. In some embodiments, S1 includes 10 or more ¹⁰B isotopes, ¹¹B isotopes, or combinations thereof. In some embodiments, S1 includes one or more carboranes. In some embodiments, S1 includes 1-amido-1-carbadodecaborate. In some embodiments, S1 includes 1-(2-amidoethyl)-3-(dodecaborate-1-thio)pyrrolidine-2,5-dione. In some embodiments, S1 includes at least one radio isotope portion. In some embodiments, the one radio isotope portion includes a chelating portion and at least one radio isotope bound to the chelating portion. In some embodiments, the radio isotope includes ⁶⁴Cu, ⁶⁷Cu, or combinations thereof.

In some embodiments, R1 is a substituted hydrocarbyl group. In some embodiments, R1 includes a hydrocarbyl group substituted with a carboxylate group, a sulfonate group, or combinations thereof.

In some embodiments, the compound has a chemical structure according to Formula IV:

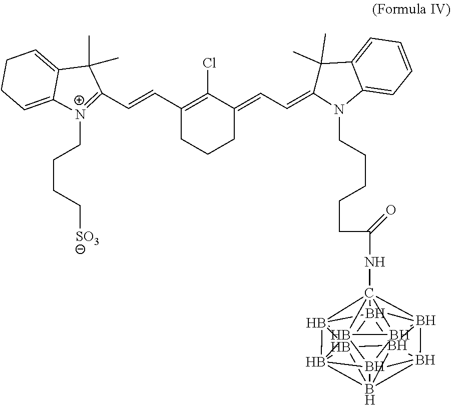

Aspects of the present disclosure are directed to a method of making a theranostic compound. In some embodiments, the method includes providing a concentration of a tumor-targeting compound, the tumor-targeting compound including a heptamethine cyanine dye (HMCD) having an available carboxyl group; reacting the available carboxyl group with ethyl chloroformate and triethylamine to form an intermediate; and reacting the intermediate with a sensitive compound including an amine and a sensitive portion to form a theranostic compound having a sensitive portion connected to a tumor targeting portion via a secondary amide. In some embodiments, the intermediate is reacted with a chelating compound including a radio isotope to attach a radio isotope portion to the intermediate prior to reacting the intermediate with the sensitive compound.

In some embodiments, the HMCD has a chemical structure according to Formula VII:

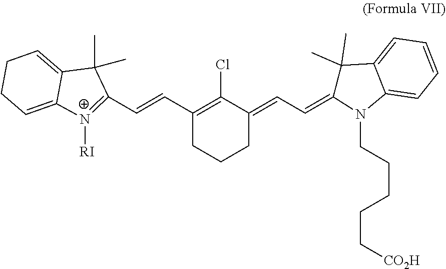

In some embodiments, the sensitive portion includes a plurality of ¹⁰B isotopes, ¹¹B isotopes, or combinations thereof. In some embodiments, the sensitive compound includes 1-amino-1-carbadodecaborate. In some embodiments, the sensitive portion includes 1-(2-amidoethyl)-3-(dodecaborate-1-thio)pyrrolidine-2,5-dione. In some embodiments, R1 is a substituted hydrocarbyl group. In some embodiments, R1 includes a hydrocarbyl group substituted with a carboxylate group, sulfonate group, or combinations thereof. In some embodiments, the theranostic compound has a chemical structure according to Formula IV.

Aspects of the present disclosure are directed to a method of treating cancer in a patient. In some embodiments, the method includes identifying cancerous tumor cells in a patient; administering to the patient an effective amount of a composition for selective uptake by the cancerous tumor cells; and contacting the cancerous tumor cells with a beam of protons, neutrons, or combinations thereof. In some embodiments, the composition includes a compound according to Formula III. In some embodiments, the compound has a chemical structure according to Formula IV.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a chart of a method of making a theranostic compound according to some embodiments of the present disclosure;

FIG. 2 is a flowchart showing a chemical synthesis scheme for theranostic compounds according to some embodiments of the present disclosure;

FIG. 3 is a chart of a method of treating cancer in a patient according to some embodiments of the present disclosure;

FIG. 4 is a flowchart showing the principle of proton capture by 11-boron isotopes and the creation of lethal alpha particles;

FIG. 5A is a graph showing analytical high-performance liquid chromatography (HPLC) data for theranostic compounds according to some embodiments of the present disclosure collected at 780 nm absorbance;

FIG. 5B is a graph showing 1H decoupled ¹¹B-NMR spectra of theranostic compounds according to some embodiments of the present disclosure using BF₃ OEt₂ as internal reference;

FIG. 5C is a graph showing mass spectral data of theranostic compounds according to some embodiments of the present disclosure indicating molecular composition and molecular weight;

FIG. 6A is an image showing LICOR cell uptake of theranostic compounds according to some embodiments of the present disclosure;

FIG. 6B is an image showing confocal fluorescence microscopy of uptake of theranostic compound according to some embodiments of the present disclosure;

FIGS. 7A-7D show graphs of MCF-7 cells grown in log phase including a control (FIG. 7A), only dye 50 μM and no theranostic compound according to embodiments of the present disclosure (FIG. 7B), with only boron ball 50 μM (FIG. 7C), and with 50 μM theranostic compound (FIG. 7D) for 4 hr. incubation, with thorough cell washing and irradiation with proton beam; and

FIG. 8 is a series of images showing a mice xenograft model of MCF-7 tumor that demonstrates the cancerous tumor cell specificity of the theranostic compounds according to embodiments of the present disclosure.

### DESCRIPTION

Some embodiments of the present disclosure are directed to compositions of proton and neutron sensitive theranostic compounds for boron proton capture therapy (BPCT), boron neutron capture therapy (BNCT), and combinations thereof. In some embodiments, the compositions include one or more theranostic compounds. In some embodiments, the compositions include two or more theranostic compounds. In some embodiments, the compositions include a plurality of theranostic compounds having different chemical structures. In some embodiments, the theranostic compounds are prodrugs, as will be discussed in greater detail below.

In some embodiments, the theranostic compound has the following general structure according to Formula I:

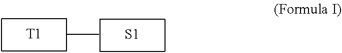

In some embodiments, the compounds include a tumor-targeting portion T1. In some embodiments, tumor-targeting portion Tl selectively binds or is taken up in cancerous tumor cells. In some embodiments, tumor-targeting portion Tl includes one or more dye compounds. In some embodiments, tumor-targeting portion T1 includes a heptamethine cyanine dye (HMCD) or a derivative thereof. In some embodiments, tumor-targeting portion Tl includes MHI-148, MHI-148 derivatives, DZ-1, DZ-1 derivatives, or combinations thereof.

In some embodiments, the compound includes a sensitive portion S1. In some embodiments, sensitive portion S1 is a proton sensitive compound. In some embodiments, sensitive portion S1 is a neutron sensitive compound. In some embodiments, sensitive portion S1 includes 2, 3, 4, 5, 6, 7, 8, 9, 10, 11, 12, or more boron isotopes. In some embodiments, sensitive portion S1 includes one or more ¹⁰B isotopes, ¹¹B isotopes, or combinations thereof. In some embodiments, sensitive portion S1 includes a plurality of ¹⁰B isotopes, ¹¹B isotopes, or combinations thereof. In some embodiments, sensitive portion S1 includes one or more boron isotope-including compounds. In some embodiments, the boron compound includes 2, 3, 4, 5, 6, 7, 8, 9, 10, 11, 12, or more boron isotopes. In some embodiments, the boron compound includes one or more ¹⁰B isotopes, ¹¹B isotopes, or combinations thereof. In some embodiments, the boron compound includes a plurality of ¹⁰B isotopes, ¹¹B isotopes, or combinations thereof. In some embodiments, the boron compound includes a carborane, carborane derivative, or combination thereof. In some embodiments, the carboranes include one or more ¹⁰B isotopes, ¹¹B isotopes, or combinations thereof. In some embodiments, sensitive portion S1 includes one or more carboranes.

While the embodiments of the theranostic compounds described above are shown to have a tumor-targeting portion T1 and a sensitive portion S1, the present disclosure is not limiting in this regard, as embodiments of the theranostic compounds can include two or more tumor-targeting portions, two or more sensitive portions, or combinations thereof. In some embodiments, the tumor-targeting portions and sensitive portions are substituted with one or more groups such as C₁-Cₙ alkyl groups, C₂-Cₙ alkenyl groups, C₂-Cₙ alkynyl groups, C₃-Cₙ cycloalkyl groups, aryl groups, etc., or combinations thereof. In some embodiments, the one or more substituent groups themselves include one or more substitutions.

In some embodiments, the theranostic compound has the following general structure according to Formula II:

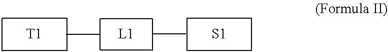

In some embodiments, one or more linkers (L1) are disposed between tumor-targeting portion T1 and sensitive portion S1. In some embodiments, linker L1 includes one or more C₁-Cₙ alkyl groups, C₂-Cₙ alkenyl groups, C₂-Cₙ alkynyl groups, C₃-Cₙ cycloalkyl groups, aryl groups, or combinations thereof. In some embodiments, the linker includes polyethylene glycol (PEG). In some embodiments, linker L1 itself includes one or more substitutions.

In some embodiments, the one or more linkers L1 include at least one radio isotope portion. In some embodiments, the one radio isotope portion includes a chelating portion and at least one radio isotope bound to the chelating portion. In some embodiments, the chelating portion includes 1,8-Diamino-3,6,10,13,16,19-hexaazabicyclo[6,6,6]-eicosane (DiAmSar), DiAmSar derivatives, 2,2′,2″-(2-(4-aminobenzyl)-1,4,7-triazonane-1,4,7-triyl)triacetic acid (p-NH2-Bn-NOTA), p-NH2-Bn-NOTA derivatives, (E)-N-(2-aminoethyl)-2-((E)-3-(2-(methylcarbamothioyl) hydrazono) butan-2-ylidene) hydrazine-1-carbothioamide (AMHC), AMHC derivatives, 1,4,7,10-Tetraazacyclododecane-1,4,7,10-tetraacetic acid (DOTA), DOTA derivatives, 4,11-bis(carboxymethyl)-1,4,8,11-tetraazabicyclo[6.6.2] hexadecane (CB-TE2A), CB-TE2A derivatives, (7R,8R)-7-(2-carboxyethyl)-5-(carboxymethyl)-18-ethyl-2,8,12,17-tetramethyl-13-vinyl-7H,8H-porphyrin-3-carboxylic acid (Ce6), Ce6 derivatives, or combinations thereof. In some embodiments, the radio isotope includes ⁶⁴Cu, ⁶⁷Cu, or combinations thereof.

In some embodiments, tumor-targeting portion Tl is directly connected to the radio isotope portion. In some embodiments, tumor-targeting portion Tl is directly connected to the radio isotope portion via at least one linker. In some embodiments, the linker is positioned between the tumor-targeting portion and the chelating portion.

In some embodiments, the theranostic compound has a chemical structure according to Formula III:

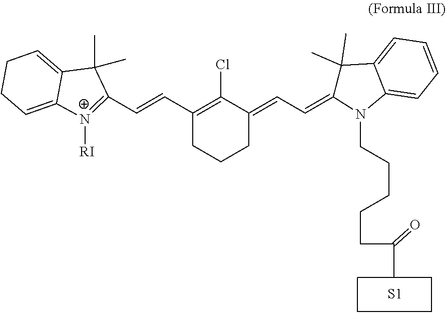

As discussed above, in some embodiments, sensitive portion S1 is a proton sensitive compound. In some embodiments, sensitive portion S1 is a neutron sensitive compound. In some embodiments, sensitive portion S1 includes 2, 3, 4, 5, 6, 7, 8, 9, 10, 11, 12, or more boron isotopes. In some embodiments, sensitive portion S1 includes one or more ¹⁰B isotopes, ¹¹B isotopes, or combinations thereof. In some embodiments, sensitive portion S1 includes a plurality of ¹⁰B isotopes, ¹¹B isotopes, or combinations thereof. In some embodiments, sensitive portion S1 includes one or more boron isotope-including compounds. In some embodiments, the boron compound includes 2, 3, 4, 5, 6, 7, 8, 9, 10, 11, 12, or more boron isotopes. In some embodiments, the boron compound includes one or more ¹⁰B isotopes, ¹¹B isotopes, or combinations thereof. In some embodiments, the boron compound includes a plurality of ¹⁰B isotopes, ¹¹B isotopes, or combinations thereof. In some embodiments, the boron compound includes a carborane, carborane derivative, or combination thereof. In some embodiments, the carboranes include one or more ¹⁰B isotopes, ¹¹B isotopes, or combinations thereof. In some embodiments, sensitive portion S1 includes one or more carboranes. In some embodiments, sensitive portion S1 includes 1-amido-1-carbadodecaborate. In some embodiments, sensitive portion S1 includes 1-(2-amidoethyl)-3-(dodecaborate-1-thio)pyrrolidine-2,5-dione.

In some embodiments, R1 is a substituted hydrocarbyl group. In some embodiments, R1 includes a hydrocarbyl group substituted with a carboxylate group, a sulfonate group, or combinations thereof.

In some embodiments, the theranostic compound has a chemical structure according to Formula IV:

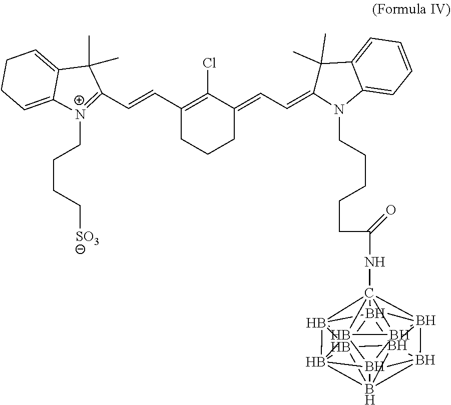

In some embodiments, the theranostic compound has a chemical structure according to Formula V:

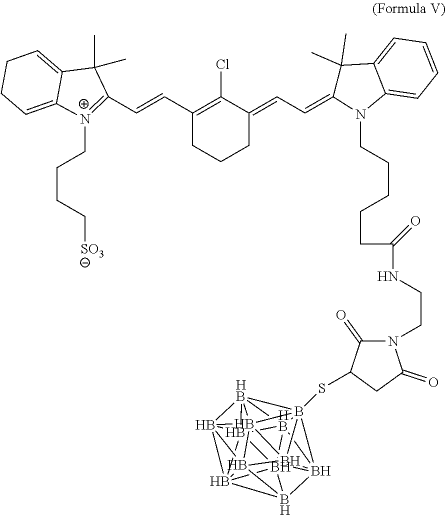

5 In some embodiments, the chemical structure according to Formula III further includes at least one radio isotope portion. In some embodiments, the one radio isotope portion is positioned between sensitive portion S1 and the neighboring carbonyl group. As discussed above, in some embodiments, the one radio isotope portion includes a chelating portion and at least one radio isotope bound to the chelating portion. In some embodiments, the chelating portion includes 1,8-Diamino-3,6,10,13,16,19-hexaazabicyclo[6,6,6]-eicosane (DiAmSar), DiAmSar derivatives, 2,2′,2″-(2-(4-aminobenzyl)-1,4,7-triazonane-1,4,7-triyl)triacetic acid (p-NH2-Bn-NOTA), p-NH2-Bn-NOTA derivatives, (E)-N-(2-aminoethyl)-2-((E)-3-(2-(methylcarbamothioyl) hydrazono) butan-2-ylidene) hydrazine-1-carbothioamide (AMHC), AMHC derivatives, 1,4,7,10-Tetraazacyclododecane-1,4,7,10-tetraacetic acid (DOTA), DOTA derivatives, 4,11-bis(carboxymethyl)-1,4,8, 11-tetraazabicyclo[6.6.2] hexadecane (CB-TE2A), CB-TE2A derivatives, (7R,8R)-7-(2-carboxyethyl)-5-(carboxymethyl)-18-ethyl-2,8,12,17-tetramethyl-13-vinyl-7H,8H-porphyrin-3-carboxylic acid (Ce6), Ce6 derivatives, or combinations thereof. In some embodiments, the radio isotope includes ⁶⁴Cu, ⁶⁷Cu, or combinations thereof. In some embodiments, the theranostic compound has a chemical structure according to Formula VI:

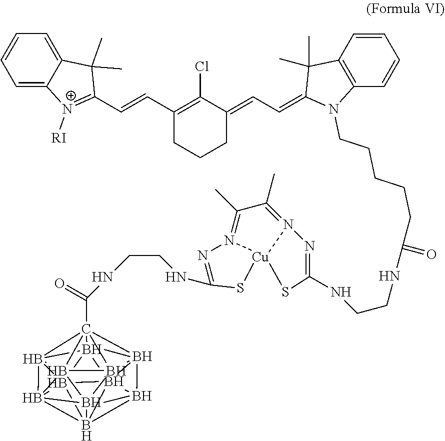

As discussed above, in some embodiments, R1 is a substituted hydrocarbyl group. In some embodiments, R1 includes a hydrocarbyl group substituted with a carboxylate group, a sulfonate group, or combinations thereof.

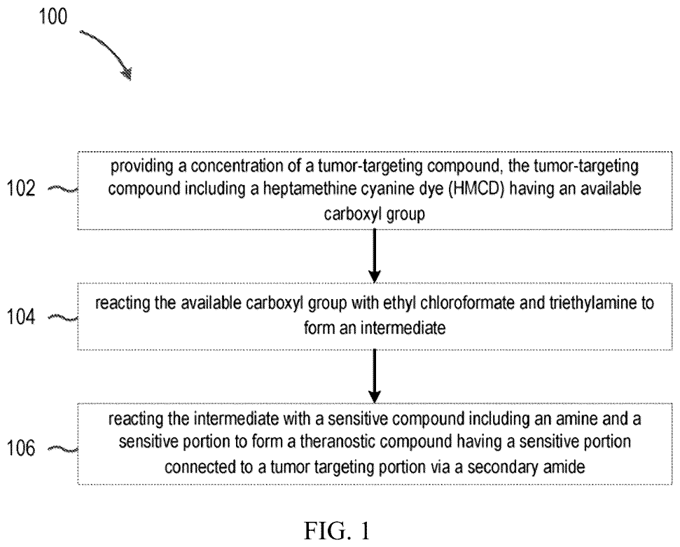

Referring now to FIG. 1, some embodiments of the present disclosure are directed to a method 100 of making a theranostic compound. At 102, a concentration of a tumor-targeting compound is provided. In some embodiments, the tumor-targeting compound is provided 102 to any suitable reaction vessel in any desired amount. In some embodiments, the tumor-targeting portion selectively binds or is taken up in cancerous tumor cells. In some embodiments, the tumor-targeting compound includes an HMCD. In some embodiments, the HMCD includes an available carboxyl group, i.e., capable of reaction with one or more additional reactants in the reaction vessel. In some embodiments, the HMCD includes MHI-148, MHI-148 derivatives, DZ-1, DZ-1 derivatives, or combinations thereof. In some embodiments, the HMCD has a chemical structure according to Formula VII:

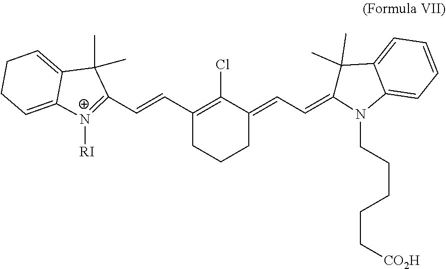

As discussed above, in some embodiments, R1 is a substituted hydrocarbyl group. In some embodiments, R1 includes a hydrocarbyl group substituted with a carboxylate group, a sulfonate group, or combinations thereof.

In some embodiments, at 104, the available carboxyl group is reacted to form an intermediate. In some embodiments, the available carboxyl group is reacted 104 with ethyl chloroformate and triethylamine to form the intermediate. The ethyl chloroformate and triethylamine reactants attack the carboxyl group and advantageously activate it for facile formation of secondary amides and/or connection of pendent compounds via secondary amide linking groups. In some embodiments, the intermediate has a chemical structure according to Formula VIII:

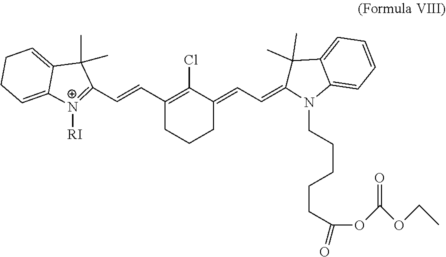

In some embodiments, at 106, the intermediate is reacted with a sensitive compound to form a theranostic compound having a sensitive portion connected to a tumor targeting portion. In some embodiments, the sensitive compound includes an amine and a sensitive portion. In some embodiments, the sensitive portion is connected to a tumor targeting portion via a secondary amide.

In some embodiments, the sensitive portion includes 2, 3, 4, 5, 6, 7, 8, 9, 10, 11, 12, or more boron isotopes. In some embodiments, the sensitive portion includes one or more ¹⁰B isotopes, ¹¹B isotopes, or combinations thereof. In some embodiments, the sensitive portion includes a plurality of ¹⁰B isotopes, ¹¹B isotopes, or combinations thereof. In some embodiments, the sensitive portion includes one or more boron isotope-including compounds. In some embodiments, the boron compound includes 2, 3, 4, 5, 6, 7, 8, 9, 10, 11, 12, or more boron isotopes. In some embodiments, the boron compound includes one or more ¹⁰B isotopes, ¹¹B isotopes, or combinations thereof. In some embodiments, the boron compound includes a plurality of ¹⁰B isotopes, ¹¹B isotopes, or combinations thereof. In some embodiments, the boron compound includes a carborane, carborane derivative, or combination thereof. In some embodiments, the carboranes include one or more ¹⁰B isotopes, ¹¹B isotopes, or combinations thereof. In some embodiments, sensitive portion S1 includes one or more carboranes. In some embodiments, the sensitive compound includes 1-amino-1-carbadodecaborate.

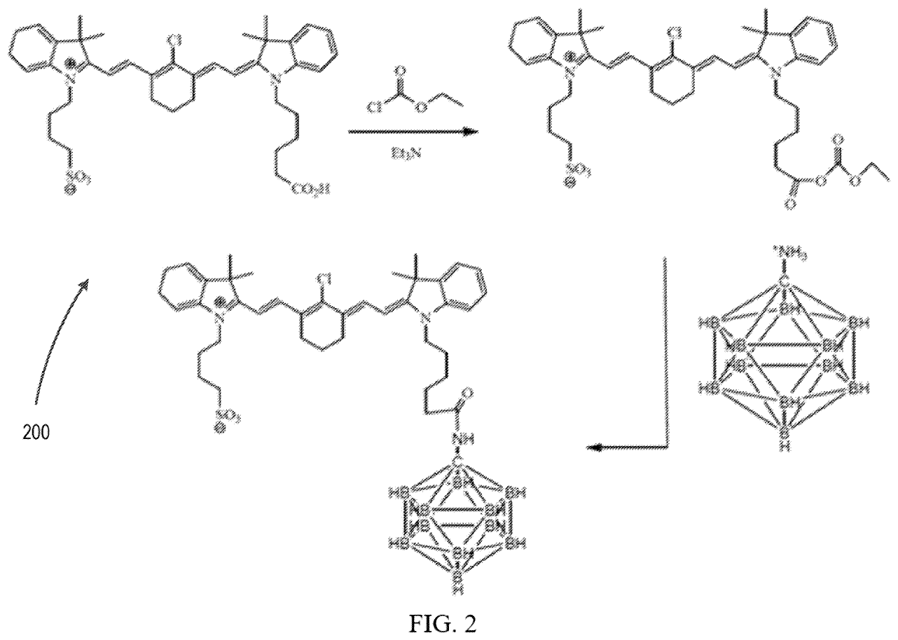

As discussed above, in some embodiments, the theranostic compound has a chemical structure according to Formula IV. An exemplary reaction scheme 200 consistent with embodiments of the present disclosure can be found at FIG. 2.

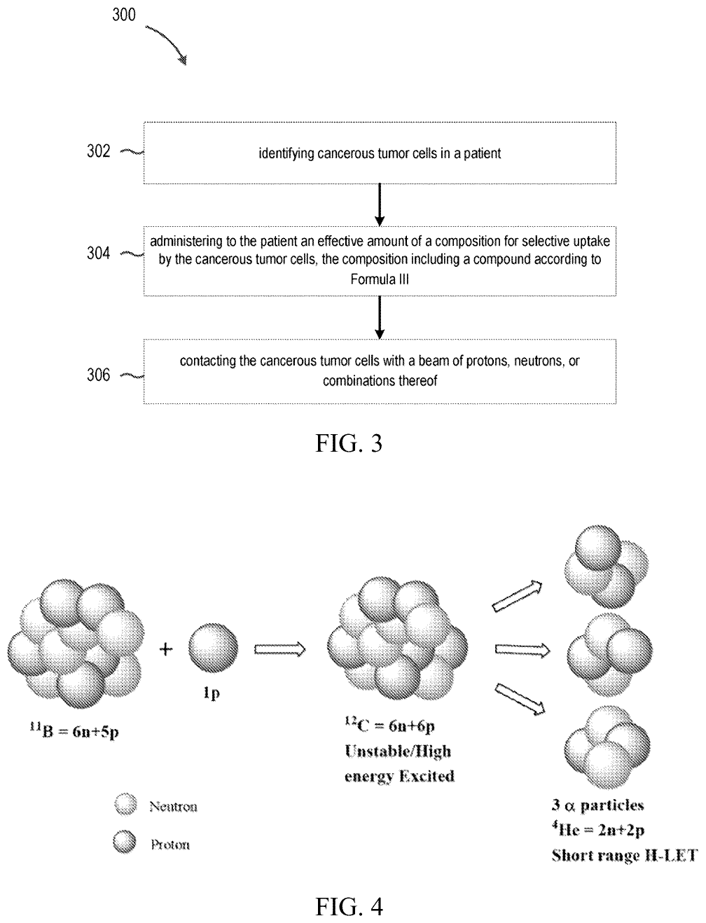

Referring now to FIG. 3, some embodiments of the present disclosure are directed to a method 300 of treating cancer in a patient. At 302, cancerous tumor cells are identified in a patient via any suitable means, e.g., via imaging, tissue analysis, etc., or combinations thereof. The types of cancer typically subjected to proton beam therapy are encompassed by the methods of the present disclosure and include, but are not limited to, brain, breast, bone, gastrointestinal tract, prostate, pediatric tumors and cancer cells, etc. At 304, an effective amount of a composition is administered to the patient. In some embodiments, the composition includes one or more proton-sensitive compounds, neutron-sensitive compounds, or combinations thereof. In some embodiments, the composition includes one or more theranostic compounds. In some embodiments, the composition includes two or more theranostic compounds. In some embodiments, the composition includes a compound according to Formula III:

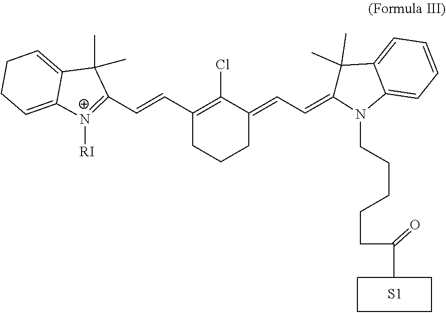

As discussed above, in some embodiments, sensitive portion S1 is a proton sensitive compound. In some embodiments, sensitive portion S1 is a neutron sensitive compound. In some embodiments, sensitive portion S1 includes 2, 3, 4, 5, 6, 7, 8, 9, 10, 11, 12, or more boron isotopes. In some embodiments, sensitive portion S1 includes one or more ¹⁰B isotopes, ¹¹B isotopes, or combinations thereof. In some embodiments, sensitive portion S1 includes a plurality of ¹⁰B isotopes, ¹¹B isotopes, or combinations thereof. In some embodiments, sensitive portion S1 includes one or more boron isotope-including compounds. In some embodiments, the boron compound includes 2, 3, 4, 5, 6, 7, 8, 9, 10, 11, 12, or more boron isotopes. In some embodiments, the boron compound includes one or more ¹⁰B isotopes, ¹¹B isotopes, or combinations thereof. In some embodiments, the boron compound includes a plurality of ¹⁰B isotopes, ¹¹B isotopes, or combinations thereof. In some embodiments, the boron compound includes a carborane, carborane derivative, or combination thereof. In some embodiments, the carboranes include one or more ¹⁰B isotopes, ¹¹B isotopes, or combinations thereof. In some embodiments, sensitive portion S1 includes one or more carboranes. In some embodiments, sensitive portion S1 includes 1-amido-1-carbadodecaborate. In some embodiments, sensitive portion S1 includes 1-(2-amidoethyl)-3-(dodecaborate-1-thio)pyrrolidine-2,5-dione.

In some embodiments, sensitive portion S1 includes at least one radio isotope portion, the one radio isotope portion including a chelating portion and at least one radio isotope bound to the chelating portion. In some embodiments, the chelating portion includes 1,8-Diamino-3,6,10,13,16,19-hexaazabicyclo[6,6,6]-eicosane (DiAmSar), DiAmSar derivatives, 2,2′,2″-(2-(4-aminobenzyl)-1,4,7-triazonane-1,4,7-triyl)triacetic acid (p-NH2-Bn-NOTA), p-NH2-Bn-NOTA derivatives, (E)-N-(2-aminoethyl)-2-((E)-3-(2-(methylcarbamothioyl) hydrazono) butan-2-ylidene) hydrazine-1-carbothioamide (AMHC), AMHC derivatives, 1,4,7,10-Tetraazacyclododecane-1,4,7,10-tetraacetic acid (DOTA), DOTA derivatives, 4,11-bis(carboxymethyl)-1,4,8, 11-tetraazabicyclo[6.6.2] hexadecane (CB-TE2A), CB-TE2A derivatives, (7R,8R)-7-(2-carboxyethyl)-5-(carboxymethyl)-18-ethyl-2,8,12,17-tetramethyl-13-vinyl-7H,8H-porphyrin-3-carboxylic acid (Ce6), Ce6 derivatives, or combinations thereof. In some embodiments, the radio isotope includes ⁶⁴Cu, ⁶⁷Cu, or combinations thereof.

In some embodiments, R1 is a substituted hydrocarbyl group. In some embodiments, R1 includes a hydrocarbyl group substituted with a carboxylate group, sulfonate group, or combinations thereof.

In some embodiments, the compound has a chemical structure according to Formula IV, Formula V, Formula VI, or combinations thereof. In some embodiments, the composition further includes a pharmaceutically acceptable buffer, diluent, carrier molecule, adjuvant, excipient, additional therapeutically active compound, or combinations thereof. In some embodiments, the composition is configured for administration intravenously, orally, etc., or combinations thereof. In some embodiments, the compound is present in the composition as a pharmaceutically-acceptable salt, e.g., of Formula III, Formula IV, Formula V, Formula VI, etc.

In some embodiments, the composition is selectively taken up by cancerous tumor cells. In some embodiments, the composition is administered 304 as a part of a process for radiotherapy, tumor imaging, or tumor growth progression analysis. Without wishing to be bound by theory, uptake and retention of the compounds of the present disclosure by cancer cells relies largely on the expression of specific organic anion transport proteins (OATPs), cell surface carriers. Unlike other approaches targeting cancer cells based on their cell surface receptors or antigens, such as PSMA antibody carriers, or gastrin receptors, the uptake and retention by multiple cancer cell types depends on the expression of a specific clusters of OATPs that transport much needed nutrients from the outer environment of the cells to inside of the cells for extensive cell growth and relatively high metabolism. OATPs expression is enhanced by hypoxia, a hallmark of a cancer cell's metabolism. Other cell surface receptor targeted imaging probes exhibit high degrees of variance in expression of receptors that are regulated by endogenous conditions such as hormones and soluble and insoluble factors in the tumor microenvironment. Other studies have led to reports that hypoxia inducing factor (HIF-1 alpha), liposomal storage, acidity of tumor environment, DNA intercalation, changed mitochondrial membrane potential, change in demand and supply of oxygen mainly due to altered metabolism of cancer cells, indicating there are additional important factors that may be responsible for the observed differential in affinity compared to normal cells. Further, once the compositions enter cancer cells, they provoke strong molecular interaction with multiple cellular machineries such as DNA and RNAs and become “trapped” in the cells, enabling retention of the composition and proton/neutron sensitive compounds in the cells for a prolonged period.

At 306, the cancerous tumor cells are contacted with a beam of protons, neutrons, or combinations thereof. In some embodiments, the beams are contacted 306 as a part of BPCT and/or BNCT. In some embodiments, proton/neutron beam contact 306 of the tumor-accumulated boronated compounds bombards the ¹⁰B and ¹¹B isotopes. Referring now to FIG. 4, the isotopes are transmutated, e.g., into three alpha particles or one alpha and one lithium-7 particles accordingly. Mechanistic studies have shown that BPCT of live cells results in damage to DNAs by these alpha particles. These high energy alpha particles travel only a short distance within cell and/or a very close tumor microenvironment, damaging cellular DNA ultimately resulting in killing cancer cells. Thus, embodiments of the present disclosure utilize the nuclear reaction of p+¹¹B→3α to enhance tumor therapy.

### EXEMPLARY EMBODIMENTS

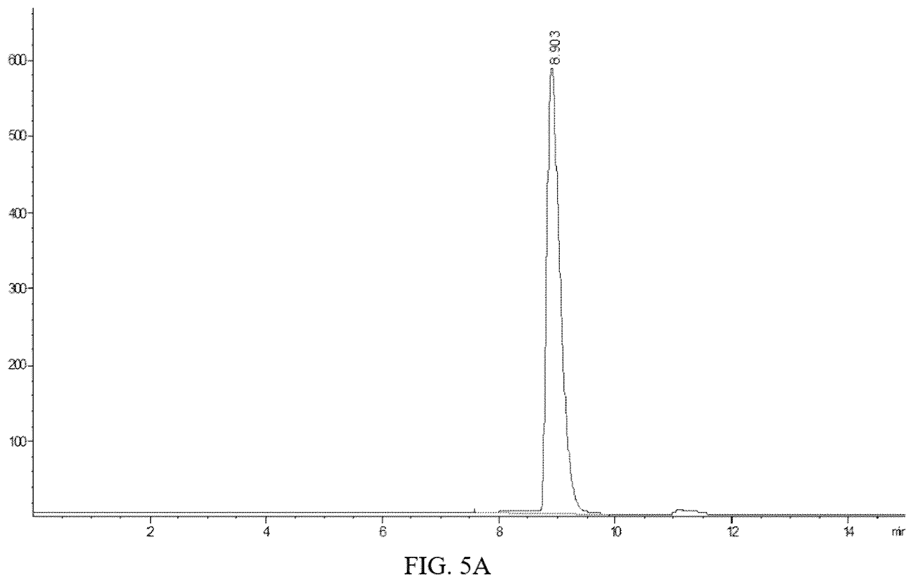

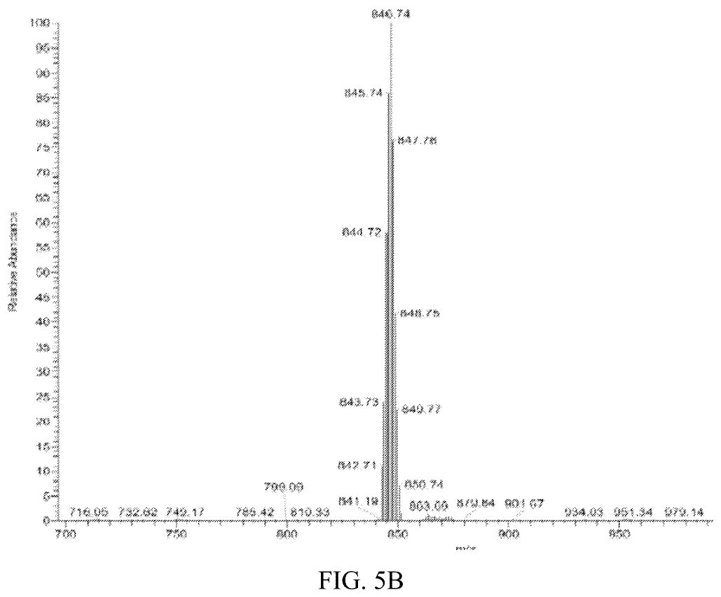

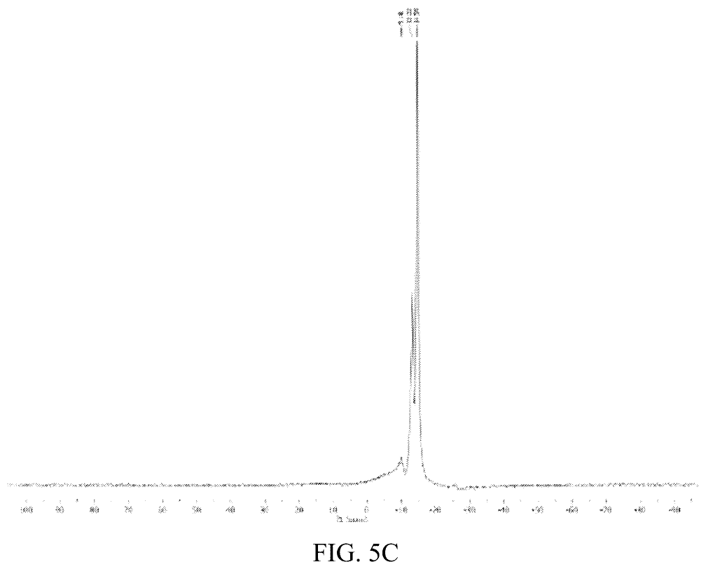

Referring now to FIGS. 5A-5C, in an exemplary embodiment to demonstrate the increase in concentration and spatial localization of boron isotopes to cancerous tissues, 1-amino-1-carba-dodecaborate was chosen for conjugation with hetero-functionalized HMCD DZ-1, having a carboxy function as a handle. The analytical purity of the compound after column chromatography was determined by analytical high-performance liquid chromatography (HPLC) (see FIG. 5A). The conjugate exhibited physico-chemical properties similar to other HMCD conjugates such as UV/Vis and fluorescence spectra, especially ¹¹B-NMR (see FIG. 5B, H-decoupled δ −14.59, −13.22 and −9.98, BF₃·OEt₂ standard) and mass (see FIG. 5C, MF: C₄₀H₆₀B₁₁ClN₃O₄S; [M]⁺ m/z 846.74 major) spectrometry analysis.

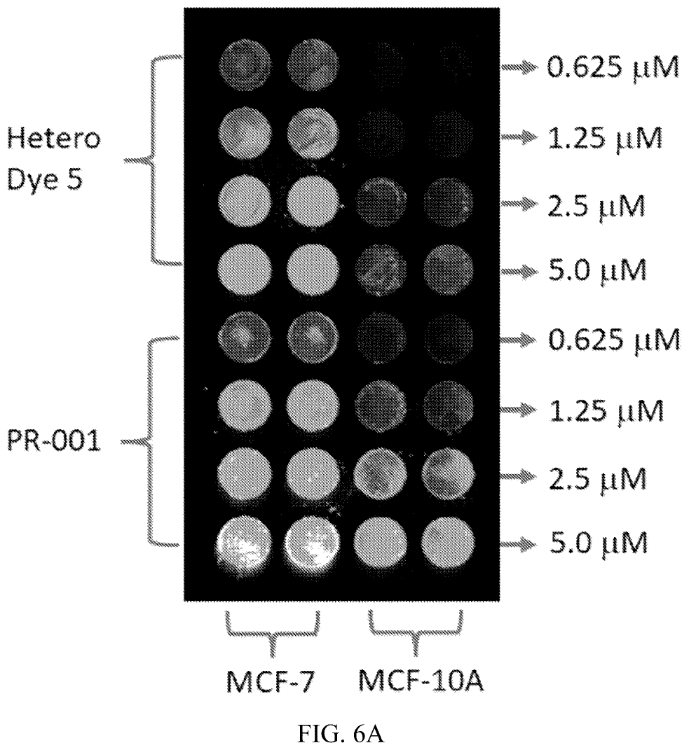

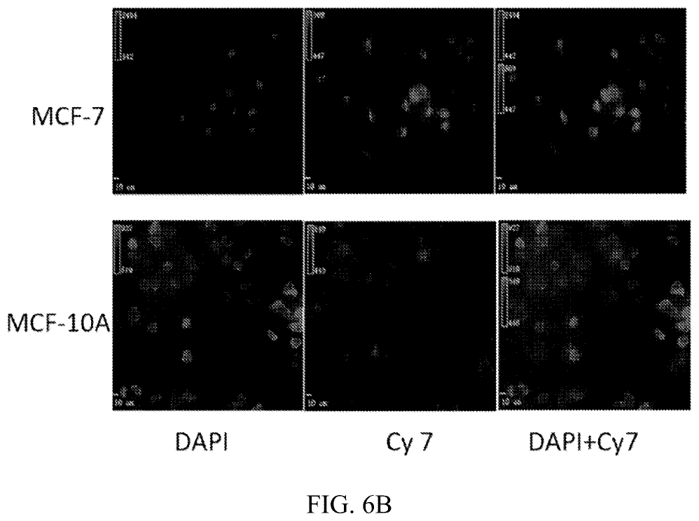

Referring now to FIGS. 6A-6B, after confirming the structure and purity of the compound, further biological evaluation was performed. Preferential cancer cell uptake was evaluated in human breast cancer cells MCF-7 compared with MCF-10A epithelial cells. Concentration dependent uptake in MCF-7 cells was observed at 0.626, 1.25 and 2.5 μM. Both parent HMCD and compounds consistent with embodiments of the present disclosure showed no or poor uptake in MCF-10A cells (as normal cell control) at all concentrations tested. The cancer cell uptake was also confirmed by confocal microscopy. The concentration and constitutional stability of the compound in cancer cells was established by lysing the MCF-7 incubated with 5 μM of compound for 4 hrs. The methanol extract of lysed cells when analyzed with thin layer chromatography and compared with compound indicated the presence of intact compound. Although the left-over cell debris has more observable green color (compound is green colored) as well as possess near infrared florescence that does not indicate the intact compound. Measuring concentration of the compound from methanolic extract by NIRF fluorescence intensity indicated ˜5-10 nM compound was present in the extract suggesting efficient amount of accumulation. Inherent toxicity of the compound to both normal and cancer cells was evaluated using HMCD and 1-amino-1-carbadodecaborate as controls. By assay, it was observed that compound at concentrations of 50-100 μM for 48 hr. incubation does not seem to cause cell death.

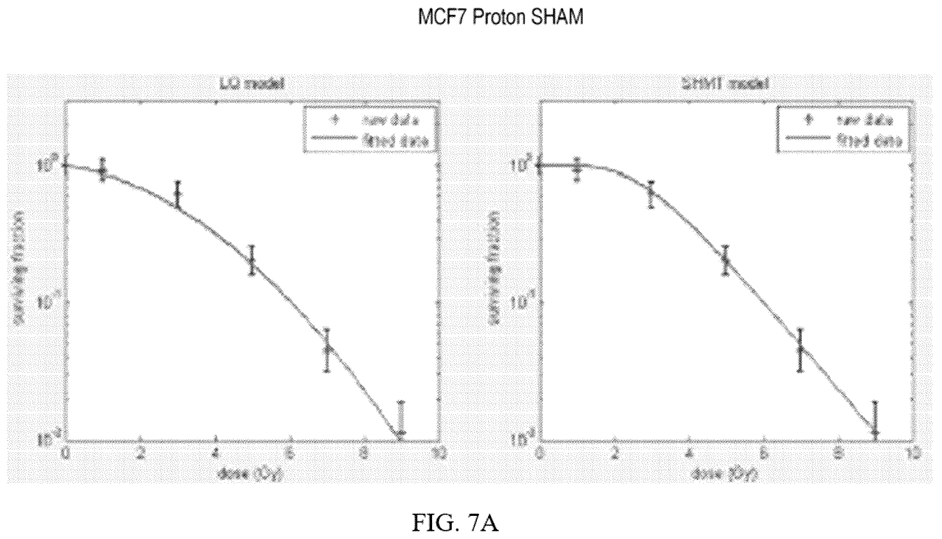

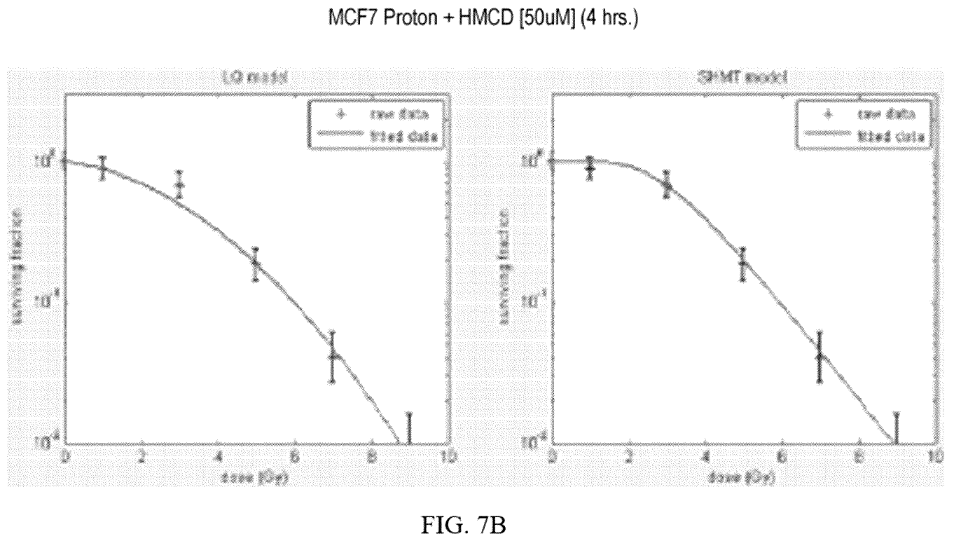

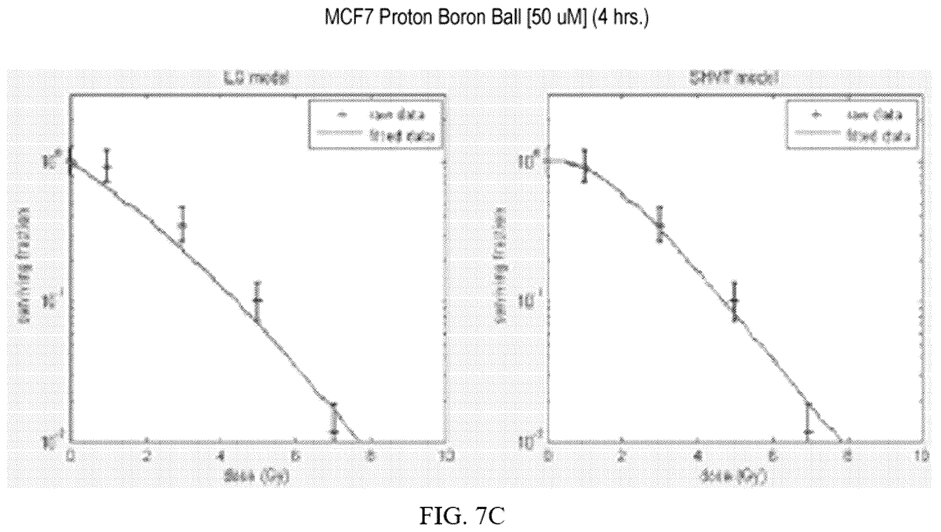

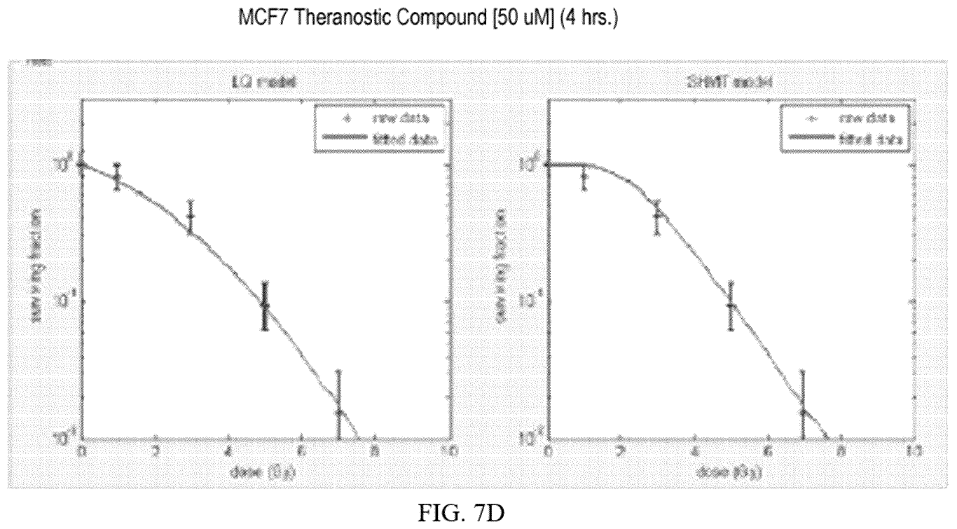

Referring now to FIGS. 7A-7D, having established uptake and accumulation of compounds consistent with embodiments of the present disclosure in cancer cells, those cells were next subjected to proton beam irradiation. MCF-7 cells were provided in four groups: 1) Only media/control; 2) HMCD only; 3) 1-amino-1-carbadodecaborate; and 4) theranostic compound consistent with embodiments of the present disclosure. Cells were counted, seeded, and grown in their respective media in T25 flasks, prior to incubation with compounds. Incubation with respective compound (50 μM each) in all groups for 4 hrs. followed by irradiation with graded proton doses of 0, 1, 3, 5,7 and 9 Gy in triplicate resulted in irradiated cells, which were seeded and incubated at 37° C. in the presence of 5% CO₂ for 10-14 days. The colonies formed were measured by standard cell survival program to obtain corresponding Do values. Based on observed results, MCF-7 cells were sensitized towards proton beam irradiation when compared with the other groups.

Changes in MCF7-cell survival after proton irradiation were as follows: MCF7 cells without theranostic compound (control) D₀=1.3 Gy, while MCF7 cells incubated with theranostic compound at 50 μM concentration for 4 hr. resulted in D₀=1.1 Gy. When the concentration of theranostic compound increased to 70 μM and was incubated for 48 hrs. prior to proton irradiation, the results were D₀=0.89 Gy. A reduction in Do of MCF cells with theranostic compound in comparison with control cells indicates effectiveness of proton beam irradiation in cancer cell killing, e.g., by lethal alpha particles released from nuclear proton capture by the 11-boron atom.

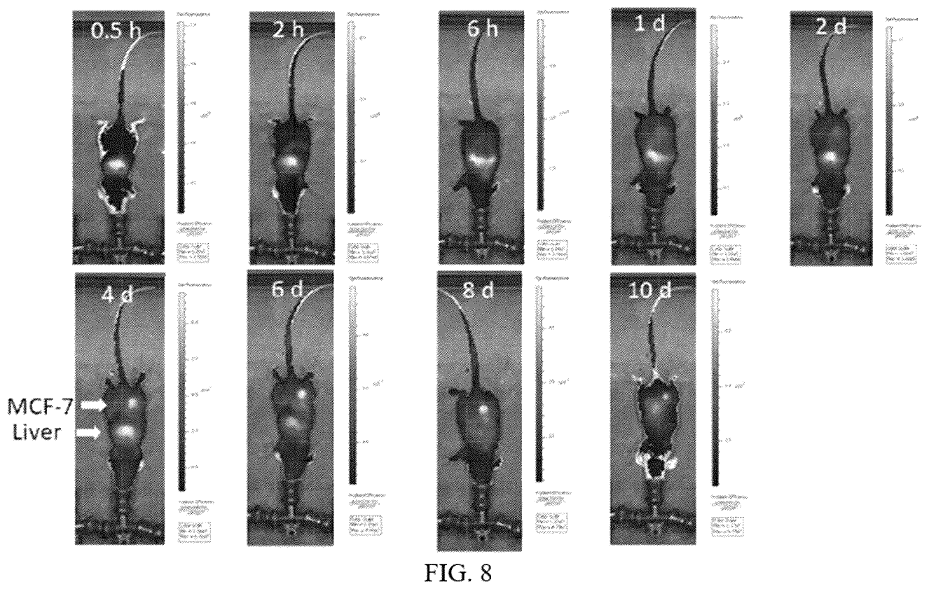

Referring now to FIG. 8, mice xenograft model of MCF-7 tumor was used to demonstrate the cancer cell targeted, tumor specific delivery of theranostic compounds according to embodiments of the present disclosure. Mice carrying MCF-7 mammary pad xenografts were intravenously injected with 100 μL (50 μM) solution of compound and serially imaged via NIRF in vivo imaging over 10 days. The mice showed time dependent distribution of theranostic compound, with the first few days (up to ˜6 days) showing uptake into the liver, followed by subsequent uptake into the tumor (4 days onwards). The liver uptake was subsided with time however, tumor uptake was retained from days 6 through 10.

Methods and systems of the present disclosure are advantageous to provide a series of boronated prodrugs including a cancer-targeting moiety, e.g., an HMCD, carrying a plurality of boron isotopes. Boron-HMCD conjugates are preferentially delivered to cancer cells verses normal cells. In an exemplary embodiment, 1-amino-1-carbadodecaborate with HMCD was used to deliver eleven ¹¹B atoms per dye molecule selectively to breast cancer cells in vitro. The boronated structures are advantageously small molecules that can target cancers for BPCT and BNCT applications, e.g., through OATPs and other cancer specific mechanisms. Furthermore, a copper radio isotope entity can be inserted into the boronated compounds.

Many other drug-HMCD conjugates which are delivered into cancer cells either release the effective drug from HMCD or retain the interaction of the conjugate with a target biomolecule in order to have any therapeutic effect. The theranostic compounds of the present disclosure need not be activated or interacting with any particular target, yet can still remain accumulated at high concentrations in tumor cells. Upon irradiation with proton or neutron beams and subsequent nuclear fusion reaction by proton capture, unstable ¹²C is generated in place of ¹¹B, which is short lived and releases three alpha particles while undergoing fission. Since embodiments of the theranostic compounds include multiple boron atoms, are selectively delivered into cancer cells, and do not exhibit any apparent toxicity, they significantly enhance the effectiveness of BPCT and BNCT and cancer treatment overall, especially in cases such as with young patients and with deep tissue tumors located in critical organs which are difficult to remove by surgery.

### Definitions

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used.

The terms “additional therapeutically active compound” or “additional therapeutic agent”, as used in the context of the present disclosure, refers to the use or administration of a compound for an additional therapeutic use for a particular injury, disease, or disorder being treated. Such a compound, for example, could include one being used to treat the same disease or disorder, or an unrelated disease or disorder, or a disease or disorder which may not be responsive to the primary treatment for the injury, disease or disorder being treated.

As used herein, the term “adjuvant” refers to a substance that elicits an enhanced immune response when used in combination with a specific antigen.

As used herein, the terms “administration of” and or “administering” a compound should be understood to mean providing a compound according to embodiments of the present disclosure or a prodrug of a compound according to embodiments of the present disclosure to a subject in need of treatment.

As used herein, an “analog”, or “analogue”, of a chemical compound is a compound that, by way of example, resembles another in structure but is not necessarily an isomer.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

As used herein, the term “carrier molecule” refers to any molecule that is chemically conjugated to the antigen of interest that enables an immune response resulting in antibodies specific to the native antigen.

A “compound”, as used herein, refers to any type of substance or agent that is commonly considered a drug, or a candidate for use as a drug, as well as combinations and mixtures of the above. When referring to a compound according to embodiments of the present disclosure, and unless otherwise specified, the term “compound” is intended to encompass not only the specified molecular entity but also its pharmaceutically acceptable, pharmacologically active analogs, including, but not limited to, salts, polymorphs, esters, amides, prodrugs, adducts, conjugates, active metabolites, and the like, where such modifications to the molecular entity are appropriate.

As used herein, a “derivative” of a compound refers to a chemical compound that may be produced from another compound of similar structure in one or more steps, as in replacement of H by an alkyl, acyl, or amino group.

As used herein, an “effective amount” or “therapeutically effective amount” means an amount sufficient to produce a desired effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary.

As used herein, the term “linker,” in some embodiments, refers to a molecule or molecules that joins two other molecules either covalently or noncovalently, e.g., through ionic or hydrogen bonds or van der Waals interactions.

As used herein, the term “pharmaceutically acceptable” means physiologically tolerable, for either human or veterinary application.

As used herein, the term “hydrocarbyl” as used herein refers to a branched or unbranched group including carbon and hydrogen, e.g., alkyl, alkenyl, alkynyl, cycloalkyl, aryl group, etc., or combinations thereof.

As used herein, the term “C₁-Cₙ alkyl” wherein n is an integer, represents a branched or linear alkyl group having from one to the specified number of carbon atoms. Examples of such groups include, but are not limited to, methyl, ethyl, n-propyl, iso-propyl, butyl, iso-butyl, sec-butyl, tert-butyl, pentyl, hexyl, and the like.

As used herein, the term “C₂-Cₙ alkenyl” wherein n is an integer, represents an olefinically unsaturated branched or linear group having from two to the specified number of carbon atoms and at least one double bond. Examples of such groups include, but are not limited to, 1-propenyl, 2-propenyl, 1,3-butadienyl, 1-butenyl, hexenyl, pentenyl, and the like.

As used herein, the term “C₂-Cₙ alkynyl” wherein n is an integer refers to an unsaturated branched or linear group having from two to the specified number of carbon atoms and at least one triple bond. Examples of such groups include, but are not limited to, 1-propynyl, 2-propynyl, 1-butynyl, 2-butynyl, 1-pentynyl, and the like.

As used herein, the term “C₃-Cₙ cycloalkyl” wherein n=8, represents cyclopropyl, cyclobutyl, cyclopentyl, cyclohexyl, cycloheptyl, and cyclooctyl.

As used herein, the term “aryl” refers to an optionally substituted mono- or bicyclic carbocyclic ring system having one or two aromatic rings including, but not limited to, phenyl, benzyl, naphthyl, tetrahydronaphthyl, indanyl, indenyl, and the like.

As used herein, the term “bicyclic” represents either an unsaturated or saturated stable 7- to 12-membered bridged or fused bicyclic carbon ring. The bicyclic ring may be attached at any carbon atom which affords a stable structure. The term includes, but is not limited to, naphthyl, dicyclohexyl, dicyclohexenyl, and the like.

As used herein the term “heteroaryl” refers to an optionally substituted mono- or bicyclic carbocyclic ring system having one or two aromatic rings containing from one to three heteroatoms and includes, but is not limited to, furyl, thienyl, pyridyl and the like.

As used herein, the term “substituted” refers to inclusion of one or more substituents, wherein the substituents are each independently selected. Each of the independently selected substituents may be the same or different than other substituents.

As used herein, the term “pharmaceutically-acceptable salt” refers to salts which retain the biological effectiveness and properties of the compounds of the present disclosure and which are not biologically or otherwise undesirable.

Pharmaceutically-acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di (substituted alkyl) amines, tri (substituted alkyl) amines, alkenyl amines, dialkenyl amines, trialkenyl amines, substituted alkenyl amines, di (substituted alkenyl) amines, tri (substituted alkenyl) amines, cycloalkyl amines, di (cycloalkyl) amines, tri (cycloalkyl) amines, substituted cycloalkyl amines, disubstituted cycloalkyl amine, trisubstituted cycloalkyl amines, cycloalkenyl amines, di (cycloalkenyl) amines, tri (cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Examples of suitable amines include, by way of example only, isopropylamine, trimethyl amine, diethyl amine, tri (iso-propyl) amine, tri (n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. Other potentially useful carboxylic acid derivatives may include, by way of example, carboxylic acid amides, including carboxamides, lower alkyl carboxamides, dialkyl carboxamides, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The terminology used herein is for the purpose of describing the particular versions or embodiments only, and is not intended to limit the scope of the present disclosure. All publications mentioned herein are incorporated by reference in their entirety.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

1. A compound according to Formula III: 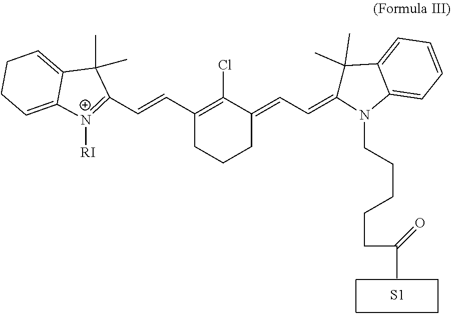
wherein
S1 includes a plurality of ¹⁰B isotopes, ¹¹B isotopes, or combinations thereof, and
R1 is a substituted hydrocarbyl group.

2. The compound according to claim 1, wherein S1 includes 10 of more ¹⁰B isotopes, ¹¹B isotopes, or combinations thereof.

3. The compound according to claim 2, wherein S1 includes one or more carboranes.

4. The compound according to claim 3, wherein S1 includes at least one of 1-amido-1-carbadodecaborate and 1-(2-amidoethyl)-3-(dodecaborate-1-thio)pyrrolidine-2,5-dione.

5. (canceled)

6. The compound according to claim 1, wherein S1 includes at least one radio isotope portion.

7. The compound according to claim 6, wherein the one radio isotope portion includes a chelating portion and at least one radio isotope bound to the chelating portion.

8. The compound according to claim 7, wherein the radio isotope includes ⁶⁴Cu, ⁶⁷Cu, or combinations thereof.

9. The compound according to claim 1, wherein R1 includes a hydrocarbyl group substituted with a carboxylate group, a sulfonate group, or combinations thereof.

10. The compound according to claim 1, wherein the compound has a chemical structure according to Formula IV:

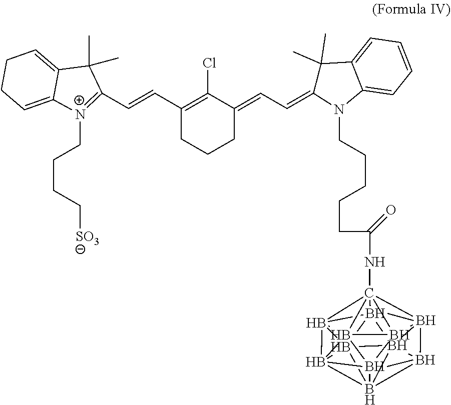

11. A method of making a theranostic compound, comprising:
providing a concentration of a tumor-targeting compound, the tumor-targeting compound including a heptamethine cyanine dye (HMCD) having an available carboxyl group;
reacting the available carboxyl group with ethyl chloroformate and triethylamine to form an intermediate; and
reacting the intermediate with a sensitive compound including an amine and a sensitive portion to form a theranostic compound having a sensitive portion connected to a tumor targeting portion via a secondary amide,
wherein the sensitive portion includes a plurality of ¹⁰B isotopes, ¹¹B isotopes, or combinations thereof.

12. The method according to claim 11, wherein the HMCD has a chemical structure according to Formula VII: 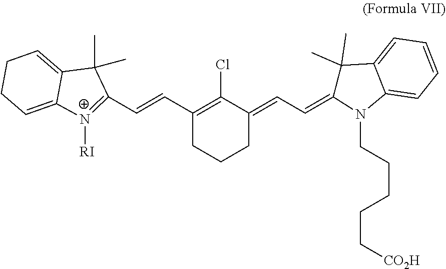
wherein R1 is a substituted hydrocarbyl group.

13. The method according to claim 12, wherein R1 includes a hydrocarbyl group substituted with a carboxylate group, sulfonate group, or combinations thereof.

14. The method according to claim 11, wherein the sensitive compound includes 1-amino-1-carbadodecaborate.

15. The method according to claim 14, wherein the theranostic compound has a chemical structure according to Formula IV:

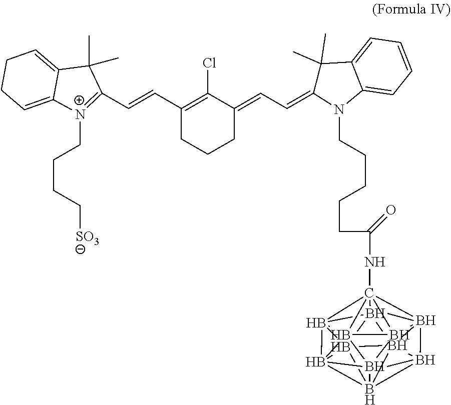

16. The method according to claim 11, wherein the intermediate is reacted with a chelating compound including a radio isotope to attach a radio isotope portion to the intermediate prior to reacting the intermediate with the sensitive compound.

17. A method of treating cancer in a patient, comprising:
identifying cancerous tumor cells in a patient;
administering to the patient an effective amount of a composition for selective uptake by the cancerous tumor cells, the composition including a compound according to Formula III: 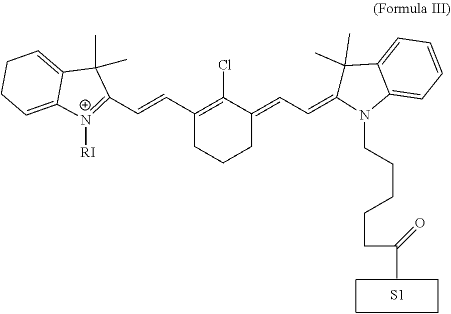
and,
contacting the cancerous tumor cells with a beam of protons, neutrons, or combinations thereof,
wherein
S1 includes a plurality of ¹⁰B isotopes, ¹¹B isotopes, or combinations thereof, and
R1 is a substituted hydrocarbyl group.

18. The method according to claim 17, wherein S1 includes at least one of 1-amido-1-carbadodecaborate and 1-(2-amidoethyl)-3-(dodecaborate-1-thio)pyrrolidine-2,5-dione.

19. (canceled)

20. The method according to claim 17, wherein R1 includes a hydrocarbyl group substituted with a carboxylate group, sulfonate group, or combinations thereof.

21. The method according to claim 17, wherein S1 includes at least one radio isotope portion, the one radio isotope portion includes a chelating portion and at least one radio isotope bound to the chelating portion, wherein the radio isotope includes ⁶⁴Cu, ⁶⁷Cu, or combinations thereof.

22. The method according to claim 17, wherein the compound has a chemical structure according to Formula IV: [chemical structure]
